(19) United States
(12) Patent Application Publication
(10) Pub. No.: US 2021/0275449 A1
(43) Pub. Date: Sep. 9, 2021

(54) Inhalable Formulation of a Solution Containing Glycopyrronium Bromide

(71) Applicants: Cai Gu Huang, Shrewsbury, MA (US); Lu Lu Zhang, Shanghai (CN)

(72) Inventors: Cai Gu Huang, Shrewsbury, MA (US); Lu Lu Zhang, Shanghai (CN)

(73) Assignee: Cai Gu Huang, Shrewsbury, MA (US)

(21) Appl. No.: 17/186,561

(22) Filed: Feb. 26, 2021

Related U.S. Application Data
(60) Provisional application No. 62/983,738, filed on Mar. 1, 2020.

(51) Int. Cl. A61K 9/00; A61K 31/40; A61K 47/18; A61K 47/12; A61M 11/04

(52) U.S. Cl. CPC A61K 9/0078; A61K 31/40; A61M 11/04; A61K 47/186; A61K 47/12; A61K 47/183

(57) ABSTRACT

The present invention discloses a liquid pharmaceutical formulation and a method for administering the liquid pharmaceutical formulation by nebulizing the pharmaceutical formulation in an inhaler. The propellant-free pharmaceutical formulation comprises: (a) glycopyrronium bromide or a salt thereof; (b) a pharmacologically acceptable preservative, (c) a pharmacologically acceptable stabilizer, (d) a solvent, (e) a pH adjusting agent, and optionally (f) other pharmaceutically acceptable additives.

### PRIORITY STATEMENT

This application claims the benefit of the filing date of U.S. Provisional Patent Application No. 62/983,738, filed on Mar. 1, 2020, the contents of which are incorporated herein by reference in its entirety.

### FIELD OF THE INVENTION

Glycopyrronium bromide (also known as glycopyrrolate), chemically known as (3RS)-3-[(2 SR)-(2-cyclopentyl-2-hydroxy-2-penylacetyl)oxy]-1,1-dimethlypyrrolidinium bromide, has the following chemical structure:

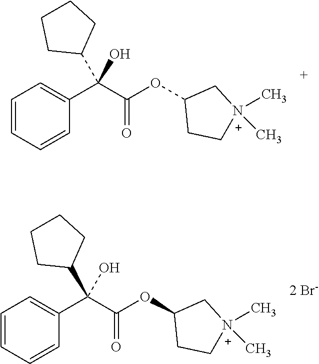

Glycopyrrolate is a long-acting muscarinic antagonist, which is often referred to as an anticholinergic, approved for the long-term maintenance treatment of airflow obstruction in patients with chronic obstructive pulmonary disease (COPD), including chronic bronchitis and/or emphysema. In chronic obstructive pulmonary disease, acetylcholine is released to airway smooth muscle and acts reversibly through postsynaptic muscarinic receptors to mediate airway smooth muscle contraction and mucus secretion. Inhaled anticholinergic agents can block muscarinic receptors on airway smooth muscle to inhibit bronchoconstriction.

Glycopyrronium bromide can provide therapeutic benefits for the treatment of asthma and chronic obstructive pulmonary disease, including chronic bronchitis and emphysema.

The present invention relates to a propellant-free inhalable formulation of glycopyrronium or its pharmaceutically acceptable salt, dissolved in water, in conjunction with inactive ingredients including a pH-adjusting agent, which can be administered by a soft mist inhalation device, and propellant-free inhalable aerosols resulting therefrom.

The pharmaceutical formulations of the present invention are particularly suitable for administering the active ingredient by soft mist inhalation, which have good lung deposition (typically up to 55-60%), compared to dry powder inhalation methods. Furthermore, the liquid inhalation formulations are advantageous compared to dry powder inhalation. In particular, administration by dry powder inhalation is more difficult, particularly for children and elderly patients.

The present invention relates to a novel approach to more effectively and selectively delivering glycopyrronium bromide to the lungs as compared to dry powder inhalation. Moreover, the pharmaceutical preparation of the current invention has a better stability as well as higher deposition in the lungs.

The pharmaceutical formulations of the present invention are particularly suitable for administering active substances by soft mist inhalation, and are particularly useful for treating asthma and chronic obstructive pulmonary disease.

### SUMMARY OF THE INVENTION

A novel, surprising approach to a more effective and selective method of delivering glycopyrronium bromide to the lungs has been found, which more effectively deposit the active ingredient in the lungs. The novel method of the current invention presents clear and significant clinical benefits, including higher efficacy and fewer adverse effects.

The present invention relates to pharmaceutical formulations of glycopyrronium or its pharmaceutically acceptable salts or solvates, which can be administered or delivered by soft mist inhalers. The pharmaceutical formulations according to the current invention meet high quality standards.

One aspect of the present invention is to provide an aqueous pharmaceutical formulation containing glycopyrronium or its pharmaceutical acceptable salts or solvates, which meets high quality standards and is able to achieve an optimum atomization effect with administration using a soft mist inhaler. It is desirable that the active ingredient in the formulation be pharmaceutically stable for a storage time period of a few months or years, such as 1-6 months, one year, or three years.

Another aspect of the present invention is providing propellant-free formulations of solutions containing glycopyrronium bromide which are nebulized under pressure using an inhaler, such as a soft mist inhaler device. In one embodiment, the formulation delivered by the inhaler is an aerosol having particle sizes that reproducibly fall within a specified and desirable range.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 shows a longitudinal section through the atomizer with stressed state;

FIG. 2 shows a counter element of the atomizer;

FIG. 3 shows the aerodynamic particle size distribution of sample VI.

FIG. 4 shows an HPLC trace demonstrating the relative retention times of impurities measured in stability experiments in Examples 6 and 8.

The use of identical or similar reference numerals in different figures denotes identical or similar features.

### DETAILED DESCRIPTION OF THE INVENTION

For purposes of describing the invention, reference now will be made in detail to embodiments and/or methods of the invention, one or more examples of which are illustrated in or with the drawings. Each example is provided by way of explanation of the invention, not limitation of the invention. In fact, it will be apparent to those skilled in the art that various modifications and variations can be made in the present invention without departing from the scope or spirit of the invention. For instance, features or steps illustrated or described as part of one embodiment, can be used with another embodiment or steps to yield a still further embodiments or methods. Thus, it is intended that the present invention covers such modifications and variations as come within the scope of the appended claims and their equivalents.

It is desirable to achieve better delivery of active substance to the lungs for the treatment of respiratory disease. Furthermore, it is desirable to maximize deposition of the drug in the lungs when the drug is delivered by inhalation.

A traditional pMDI (pressurized metered dose inhaler) or DPI (dry powder inhaler) can only deliver about 20% to about 30% of a drug into the lung, resulting in a significant amount of drug being deposited on the month and throat, which travels to the stomach and may cause unwanted side effects and/or secondary absorption through the oral digestive system.

Soft mist devices suitable for use with the aqueous pharmaceutical formulation of the present invention are those in which an amount of less than about 70 microliters of pharmaceutical solution can be nebulized in one puff, such as less than about 30 microliters, or such as less than about 15 microliters, so that the inhalable part of aerosol corresponds to a therapeutically effective quantity. In an embodiment, the average particle size of an aerosol formed from one puff is less than about 15 microns, such as less than about 10 microns.

A device of this kind for the propellant-free administration of a metered amount of a liquid pharmaceutical composition for inhalation is described in detail, for example, in U.S. 2019/0030268.

The pharmaceutical formulation in the nebulizer is converted into an aerosol destined for the lungs. The nebulizer uses high pressure to spray the pharmaceutical formulation.

In soft mist inhalers that can be used with the invention, the pharmaceutical solution is stored in a reservoir. In an embodiment, the pharmaceutical formulations the invention do not contain any ingredients which might interact with the inhaler and affect the pharmaceutical quality of the formulation or of the aerosol produced. In an embodiment, the pharmaceutical formulations of the invention are stable when stored and are capable of being administered directly.

In an embodiment, the pharmaceutical formulations of the current invention contain additives, such as the disodium salt of edetic acid (sodium edetate), to reduce the incidence of spray anomalies and to stabilize the pharmaceutical formulations. In an embodiment, the pharmaceutical formulations of the current invention have a minimum concentration of sodium edetate.

Therefore, one aspect of the present invention is to provide an aqueous pharmaceutical formulation containing glycopyrronium or its pharmaceutically acceptable salts, which meets the high standards to achieve an optimum atomization effect with administration using the inhalers mentioned hereinbefore. In one embodiment, the active substance in the pharmaceutical formulation is stable, and the pharmaceutical formulation has a storage time of some years, for example about one year, or about three years.

Another aspect of the current invention is to provide propellant-free formulations containing glycopyrronium or its pharmaceutically acceptable salts, which may be present in a solution. In an embodiment, the formulations are nebulized under pressure using an inhaler, such as a soft mist inhaler, wherein the resulting aerosol produced by the inhaler falls reproducibly within a specified range of particle size.

Another aspect of the invention is to provide an aqueous pharmaceutical formulation as a solution containing glycopyrronium or its pharmaceutically acceptable salts, and inactive excipients which can be administered by inhalation.

According to the current invention, any pharmaceutically acceptable salts or solvates of glycopyrronium may be used for the formulations. In one aspect of the invention, the pharmaceutical acceptable salt or solvate of glycopyrronium is glycopyrronium bromide.

Within the scope of the current invention, glycopyrronium bromide is preferred.

In an embodiment, the glycopyrronium bromide is dissolved in a solvent. In one embodiment, the solvent is water.

The concentration of glycopyrronium or its pharmaceutically acceptable salt in the finished pharmaceutical preparation depends on the desired therapeutic effects and can be determined by a person of ordinary skill in the art. In an embodiment, the concentration of glycopyrronium in the formulation is between about 10 mg/ml and about 6 g/100 ml, more specifically between about 100 mg/ml and about 500 mg/ml. In an embodiment, the glycopyrronium bromide is present at a concentration between about 10 mg/100 ml and about 500 mg/100 ml.

In one aspect of the invention, the formulation includes an acid or a base as pH adjusting agent. In an embodiment, the pH adjusting agent is an acid, such as citric acid and/or a salt thereof.

Other comparable pH adjusting agents can be used in the present invention. Other pH adjusting agents include, but are not limited to, hydrochloric acid and sodium hydroxide.

Adjusting the pH provides better stability of the active substance. In an embodiment, the pH ranges from about 2.0 to about 6.0. In one embodiment, the pH ranges from about 2.0 to about 5.0. In another embodiment, the pH ranges from about 2.5 to about 4.0.

In an embodiment, the pharmaceutical formulation includes a stabilizer or complexing agent. In one embodiment, the formulation includes edetic acid (EDTA) or a salt thereof, such as disodium edetate or edetate disodium dihydrate, as a stabilizer or complexing agent. In an embodiment, the formulation contains edetic acid and/or a salt thereof.

A complexing agent is a molecule which is capable of entering into complex bonds. In an embodiment, complexing agents have the effect of complexing cations. The concentration of the stabilizers or complexing agents is typically about 5 mg/100 ml to about 100 mg/100 ml. In an embodiment, the concentration of the stabilizers or complexing agents is about 10 mg/100 to about 25 mg/100 ml. In another embodiment, the concentration of the stabilizer or complexing agent is about 2 mg/100 ml to about 500 mg/100 ml. In one embodiment, the stabilizer is edetate disodium dihydrate in a concentration of about 1 mg/100 ml to about 500 mg/100 ml.

In one embodiment, the glycopyrronium bromide is present in solution.

In another embodiment, all the ingredients of the formulation are present in solution.

The formulations may further include additives. The term “additives,” as used herein, means any pharmacologically acceptable and/or therapeutically useful substance that is not an active substance, but that can be formulated together with the active substance to improve the qualities of the formulation. In an embodiment, additives have no appreciable pharmacological effect in the context of the desired therapy.

Additives include, but are not limited to, for example, other stabilizers, complexing agents, antioxidants, surfactants, preservatives which prolong the shelf life of the finished pharmaceutical formulation, vitamins, and/or other additives known in the art.

In one aspect of the invention, the formulation further comprises a suitable preservative to protect the formulation from contamination with pathogenic bacteria. In one embodiment, the preservative comprises benzalkonium chloride, benzoic acid, or sodium benzoate. In an embodiment, the pharmaceutical formulations contain only benzalkonium chloride as a preservative. In one embodiment, the amount of preservative ranges from about 2 mg/ml to about 1000 mg/100 ml. In an embodiment, the preservative is benzalkonium chloride in an amount of about 10 mg/100 ml to about 100 mg/100 ml.

To produce the propellant-free aerosols according to the invention, the pharmaceutical formulations containing glycopyrronium or its pharmaceutically acceptable salts may be used with soft mist inhaler of the kind described herein.

The soft mist inhaler disclosed in U.S. 2019/0030268, which is incorporated by reference, is an example of an inhaler that is suitable for use with the formulations of the present invention.

The soft mist inhalation device can be carried anywhere by the patient, having a cylindrical shape and convenient size of less than about 8 cm to about 18 cm long and about 2.5 cm to about 5 cm wide. The nebulizer sprays a defined volume of the pharmaceutical formulation out through small nozzles at high pressures, so as to produce inhalable aerosols.

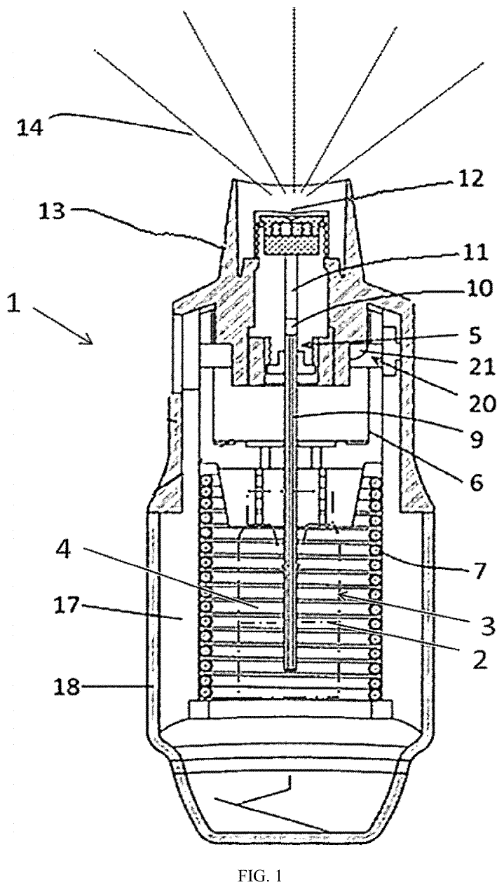

FIG. 1. shows a longitudinal section through the atomizer and a counter in the stressed state. In an embodiment, the inhalation device comprises an atomizer 1, a fluid 2, a vessel 3, a fluid compartment 4, a pressure generator 5, a holder 6, a drive spring 7, a delivering tube 9, a non-return valve 10, a pressure room 11, a nozzle 12, a mouthpiece 13, an aerosol 14, an air inlet 15, an upper shell 16, and an inside part 17.

The inhalation atomizer 1 comprising a blocking function and a counter described above for spraying a medicament fluid 2, such as a pharmaceutical formulation of the invention, is demonstrated in the FIG. 1 in the stressed state. The atomizer 1 described is a propellant-free portable inhaler.

For the typical atomizer 1 described above, an aerosol 14 that can be inhaled by a patient is generated through the atomization of the fluid 2. The pharmaceutical formulation is typically administered at least once a day, more specifically multiple times a day, such as at predetermined time gaps, according to how seriously the illness affects the patient. A person of ordinary skill in the art would be able to determine the frequency with which the pharmaceutical formulation is to be administered.

In an embodiment, the atomizer 1 described above has a substitutable and insertable vessel 3, which contains a medicament fluid 2. Therefore, a reservoir for holding the fluid 2 is formed in the vessel 3. Specifically, the medicament fluid 2 is located in the fluid compartment 4 formed by a collapsible bag in the vessel 3.

In an embodiment, the amount of fluid 2 for the atomizer 1 described above can provide an adequate amount for a patient, such as up to about 200 doses. In an embodiment, vessel 3 has a volume of about 2 to about 10 ml. A pressure generator 5 in the atomizer 1 is used to deliver and atomize the fluid 2, specifically in a predetermined dosage amount. The fluid 2 is released and sprayed in individual doses, such as from about 5 to about 30 microliters.

In an embodiment, the atomizer 1 described above may have a pressure generator 5, a holder 6, a drive spring 7, a delivering tube 9, a non-return valve 10, a pressure room 11, and a nozzle 12 in the area of a mouthpiece 13. The vessel 3 is latched by the holder 6 in the atomizer 1 so that the delivering tube 9 is plunged into the vessel 3. The vessel 3 may be separated from the atomizer 1 for substitution.

In an embodiment, when drive spring 7 is stressed in an axial direction, the delivering tube 9 and the vessel 3, along with the holder 6, will be shifted downward. The fluid 2 will be sucked into the pressure room 11 through delivering tube 9 and the non-return valve 10.

In an embodiment, after releasing the holder 6, the stress is eased. During this process, the delivering tube 9 and closed non-return valve 10 are shifted back upward by releasing the drive spring 7. The fluid 2 is under pressure in the pressure room 11. The fluid 2 is pushed through the nozzle 12 and atomized into an aerosol 14 by the pressure. A patient may inhale the aerosol 14 through the mouthpiece 13, while air is sucked into the mouthpiece 13 through air inlets 15.

In an embodiment, the atomizer 1 described above has an upper shell 16 and an inside part 17, which may be rotated relative to the upper shell 16. A lower shell 18 is manually operable to attach onto the inside part 17. The lower shell 18 may be separated from the atomizer 1 so that the vessel 3 may be substituted and inserted.

In an embodiment, the inhalation atomizer 1 described above may have a lower shell 18, which carries the inside part 17, and may be rotatable relative to the upper shell 16. As a result of rotation and engagement between the upper unit 17 and the holder 6, through a gear 20, the holder 6 is axially moved counter to the force of the drive spring 7, and the drive spring 7 is stressed.

In an embodiment, in the stressed state, the vessel 3 is shifted downwards and reaches a final position, which is demonstrated in FIG. 1. The drive spring 7 is stressed under this final position. Then the holder 6 is clasped. The vessel 3 and the delivering tube 9 are prevented from moving upwards so that the drive spring 7 is stopped from easing.

In an embodiment, in the stressed state, the atomizing process occurs after releasing the holder 6. The vessel 3, the delivering tube 9, and the holder 6 are shifted back by the drive spring 7 to the beginning position. This is referred to as major shifting. While the major shifting occurs, the non-return valve 10 is closed and the fluid 2 is under pressure in the pressure room 11 by the delivering tube 9, and then the fluid 2 is pushed out and atomized by the pressure.

In an embodiment, the inhalation atomizer 1 described above may have a clamping function. During the clamping, the vessel 3 preferably performs a lifting shift for the withdrawal of the fluid 2 during the atomizing process. The gear 20 has sliding surfaces 21 on the upper shell 16 and/or on the holder 6, which may make holder 6 axially move when the holder 6 is rotated relative to the upper shell 16.

In an embodiment, the holder 6 is not blocked for too long and can carry on the major shifting. The fluid 2 is pushed out and atomized.

In an embodiment, when the holder 6 is in the clamping position, the sliding surfaces 21 move out of engagement. The gear 20 releases the holder 6 for the opposite axial shift.

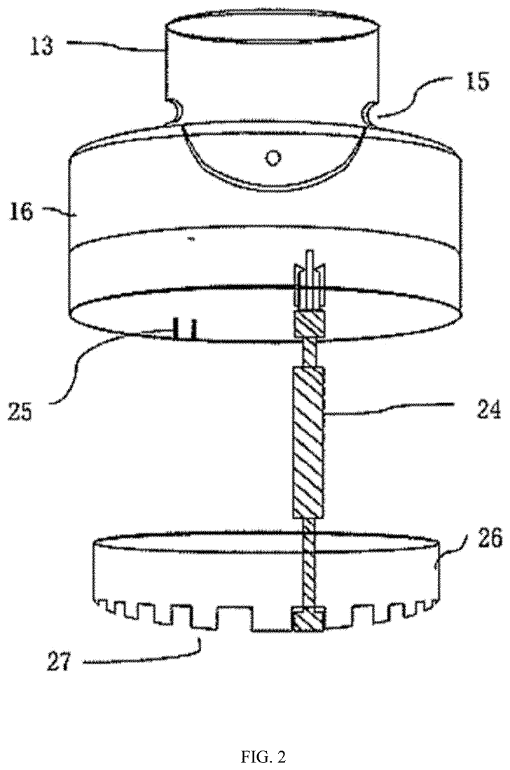

In an embodiment, the atomizer 1 includes a counter element shown in FIG. 2. The counter element has a worm 24 and a counter ring 26. In an embodiment, the counter ring 26 is circular and has a dentate part at the bottom. The worm 24 has upper and lower end gears. The upper end gear contacts with the upper shell 16. The upper shell 16 has inside bulge 25. When the atomizer 1 is employed, the upper shell 16 rotates; and when the bulge 25 passes through the upper end gear of the worm 24, the worm 24 is driven to rotate. The rotation of the worm 24 drives the rotation of the counter ring 26 through the lower end gear. This results in the counting effect.

In an embodiment, the locking mechanism is realized mainly by two protrusions. Protrusion A is located on the outer wall of the lower unit of the inside part. Protrusion B is located on the inner wall of counter. The lower unit of the inside part is nested in the counter. In an embodiment, the counter may rotate relative to the lower unit of the inside part. Because of the rotation of the counter, the number displayed on the counter may change as the actuation number increases and may be observed by the patient. After each actuation, the number displayed on the counter changes. Once the predetermined number of actuations is achieved, Protrusion A and Protrusion B will encounter each other, and the counter will be prevented from further rotation. This blocks the atomizer, stopping it from further use. The number of actuations of the device may be counted by the counter.

The nebulizer described above is suitable for nebulizing the aerosol formulations according to the invention to form an aerosol suitable for inhalation.

### EXAMPLES

Materials and Reagents:

50% benzalkonium chloride (referred to as BAC) aqueous solution is commercially available and may be purchased from Spectrum Pharmaceuticals Inc.

Edetate disodium dehydrate is commercially available and may be purchased from purchased from Merck & Co. Hydrochloric acid is also commercially available and may be purchased from Titan Reagents.

Glycopyrrolate is also commercially available and may be purchased from Nanjing Daqin Pharma Co., Ltd.

### Example 1

Sample I and Sample II inhalation solutions were prepared as follows:

50% benzalkonium chloride aqueous solution and edetate disodium dihydrate according to Table 1 were dissolved in 95 ml purified water, and the resulting solution was adjusted to the target pH with hydrochloric acid as shown in Table 1. Glycopyrronium bromide according to the amounts provided in Table 1 was added to the solution, and the resulting mixture was sonicated until completely dissolved. Finally, purified water was added to make 100 ml in volume.

TABLE 1
Ingredient Contents of Sample I And Sample II

| Ingredients | Sample I | | Sample II | |
|---|---|---|---|---|
| Glycopyrronium bromide | 113.12 | mg | 113.12 | mg |
| Edetate Disodium Dihydrate | 11 | mg | 11 | mg |
| 50% benzalkonium chloride | 20 | mg | 20 | mg |
| aqueous solution | | | | |

| | | |
|---|---|---|
| Hydrochloric acid | To pH 2.5 | To pH 3.0 |
| Purified water | Added to 100 ml | Added to 100 ml |

### Example 2

Sample III and Sample IV inhalation solutions were prepared as follows:

50% benzalkonium chloride aqueous solution and edetate disodium dihydrate according to Table 2 were dissolved in 95 ml purified water, and the resulting solution was adjusted to the target pH with hydrochloric acid as shown in Table 2. Glycopyrronium bromide according to the amounts provided in Table 2 was added to the solution, and the resulting mixture was sonicated until completely dissolved. Finally, purified water was added to make 100 ml in volume.

TABLE 2
Ingredient Contents of Sample III and Sample IV

| Ingredients | Sample III | | Sample IV | |
|---|---|---|---|---|
| Glycopyrronium bromide | 113.12 | mg | 113.12 | mg |
| Edetate Disodium Dihydrate | 11 | mg | 11 | mg |
| 50% benzalkonium chloride | 20 | mg | 20 | mg |
| aqueous solution | | | | |

| | | |
|---|---|---|
| Hydrochloric acid | To pH 3.7 | To pH 6.0 |
| Purified water | Added to 100 ml | Added to 100 ml |

### Example 3

Sample V inhalation solution was prepared as follows:

50% benzalkonium chloride aqueous solution and edetate disodium dihydrate according to Table 3 were dissolved in 95 ml purified water, and the resulting solution was adjusted to the target pH with hydrochloric acid as shown in Table 3. Glycopyrronium bromide according to the amounts provided in Table 3 was added to the solution, and the resulting mixture was sonicated until completely dissolved. Finally, purified water was added to make 100 ml in volume.

TABLE 3
Ingredient Contents of Sample V

| Ingredients | Sample V | |
|---|---|---|
| Glycopyrronium bromide | 113.12 | mg |
| Edetate Disodium Dihydrate | 11 | mg |
| 50% benzalkonium chloride | 20 | mg |
| aqueous solution | | |

| | |
|---|---|
| Hydrochloric acid | To pH 4.0 |
| Purified water | Added to 100 ml |

### Example 4

Samples I-V were sprayed using the soft mist inhalation device. A Malvern Spraytec (STP5311) instrument was used to measure the particle size of the droplets. The results are shown in Table 4.

TABLE 4
Particle Size Distribution of Samples
I-V Using A Soft Mist Inhalation Device

| | |
|---|---|
| Sample Number | Droplet size (μm) |

| | | |
|---|---|---|
| Sample I-V | D10 | 2.1 |
| | D50 | 4.0 |
| | D90 | 8.0 |

### Example 5

Aerodynamic Particle Size Distribution of Samples I-V

An Andersen Cascade Impactor instrument (ACI) was used to determine the particle size distribution of Samples I-V. The test was conducted at a flow rate of 28.3 L/min. Deposition of the active substance on each ACI plate was confirmed by high pressure liquid chromatography. The particle size was expressed in terms of mass median aerodynamic diameter (MMAD) and Geometric Standard Deviation (GSD).

TABLE 5
Aerodynamic Particle Size Distribution

| Parameter | Glycopyrronium bromide |
|---|---|
| MMAD (μm) | 4.4 |
| GSD | 1.7 |

### Example 6

Influence of pH on Stability:

The stability of glycopyrronium bromide solution is highly dependent on pH. Eight samples were prepared according to Table 6a and 6b. 50% benzalkonium chloride aqueous solution and edetate disodium dihydrate according to Table 6a and 6b were dissolved in 90 ml of purified water. Samples 1-5 were adjusted to pH 2.5, 3.0, 3.5, 4.0, and 5.0 with citric acid, respectively. Sample 6 pH was left unadjusted. Sample 7 was adjusted to pH 7 with NaOH. Sample 8 was adjusted to pH 1.3 with HCl. Glycopyrrolate according to the amounts in Table 6a and 6b was added to each solution, and the resulting mixtures were sonicated until completely dissolved. Finally, purified water was added to a final volume of 100 ml for each sample.

The formulae of the eight samples are shown in Table 6a and 6b. The preparation method is the same as Example 1. The experimental results of influencing factors are shown in Table 7.

TABLE 6a
Formulation Design of Glycopyrrolate Screening at Different pH Values

| Ingredients | Sample 1 | | Sample 2 | | Sample 3 | | Sample 4 | | Sample 5 | | Sample 6 | |
|---|---|---|---|---|---|---|---|---|---|---|---|---|
| Glycopyrrolate | 113.12 | mg | 113.12 | mg | 113.12 | mg | 113.12 | mg | 113.12 | mg | 113.12 | mg |
| 50% benzalkonium | 20.00 | mg | 20.00 | mg | 20.00 | mg | 20.00 | mg | 20.00 | mg | 20.00 | mg |
| chloride aqueous | | | | | | | | | | | | |
| solution | | | | | | | | | | | | |
| Edetate Disodium | 10.00 | mg | 10.00 | mg | 10.00 | mg | 10.00 | mg | 10.00 | mg | 10.00 | mg |
| Dihydrate | | | | | | | | | | | | |

| | | | | | | |
|---|---|---|---|---|---|---|
| Anhydrous | Adjust to | Adjust to | Adjust to | Adjust to | Adjust to | pH not |
| citric acid (CA) | pH 2.5 | pH 3.0 | pH 3.5 | pH 4.0 | pH 5.0 | adjusted |
| Purified water | Added to | Added to | Added to | Added to | Added to | Added to |
| | 100 ml | 100 ml | 100 ml | 100 ml | 100 ml | 100 ml |

TABLE 6b
Formulation Design of Glycopyrrolate
Screening at Different ph Values

| Ingredients | Sample 7 | | Sample 8 | |
|---|---|---|---|---|
| Glycopyrrolate | 113.12 | mg | 113.12 | mg |
| 50% benzalkonium chloride | 20.00 | mg | 20.00 | mg |
| aqueous solution | | | | |
| Edetate Disodium | 10.00 | mg | 10.00 | mg |
| Dihydrate | | | | |

| | | |
|---|---|---|
| HCl or NaOH | Adjust to pH 7.0 | Adjust to pH 1.3 |
| Purified water | Added to 100 ml | Added to 100 ml |

TABLE 7
Stability at Different pH Values

| | | conditions | | | | | | | |
|---|---|---|---|---|---|---|---|---|---|
| | | | | | | | pH not | | |
| | | pH 2.5 | pH 3.0 | pH 3.5 | pH 4.0 | pH 5.0 | adjusted | pH 7.0 | pH 1.3 |
| | | sample | | | | | | | |
| | | Sample 1 | Sample 2 | Sample 3 | Sample 4 | Sample 5 | Sample 6 | Sample 7 | Sample 8 |
| | | pH | | | | | | | |
| | | 2.5 | 3 | 3.5 | 4 | 5 | 5.86 | 7.0 | 1.3 |
| 0 Days | GB-J (%) | ND | ND | ND | ND | 3.77 | 4.48 | 8.42 | ND |
| 60° C. | GB-J (%) | 0.1 | 0.06 | 0.08 | 0.34 | 6.43 | 6.18 | 27.33 | 8.0 |
| 7 Days | | | | | | | | | |
| 60° C. | GB-J (%) | 0.2 | 0.13 | 0.17 | 0.69 | 6.7 | 7.16 | 60.25 | 18.2 |
| 14 Days | | | | | | | | | |

GB-J is the major degradation product of Glycopyrrolate. The chemical name of GB-J is 2-cyclopenty-2-hydroxy-2-phenylacetic acid.

The above results confirmed that the stability of the glycopyrrolate solution is highly dependent on the formulation pH. As can be seen from Table 7, the glycopyrronium bromide solution is stable at pH 2.5 to 6.0, with highest stability at pH 2.5-4.0.

### Example 7

Aerodynamic Particle Size Distribution:

TABLE 8
Ingredient Contents of Sample VI

| Ingredients | Sample VI | |
|---|---|---|
| Glycopyrronium bromide (GB) | 113.12 | mg |
| 50% benzalkonium chloride | 20.00 | mg |
| aqueous solution (50% BAC) | | |
| Edetate Disodium Dihydrate (EDTA) | 10.00 | mg |

| | |
|---|---|
| Anhydrous citric acid (CA) | Adjust to pH 3.0 |
| Purified water | Added to 100 ml |

The Preparation of Sample VI Inhalation Solution:

50% benzalkonium chloride aqueous solution and edetate disodium dihydrate according to Table 8 were dissolved in 95 ml purified water, and the solution was adjusted to the target pH with anhydrous citric acid. Glycopyrronium bromide according to Table 8 was added to the solution, and then the mixture was sonicated until completely dissolved. Finally, purified water was added to a final volume of 100 ml.

The aerodynamic particle size distribution was determined using a Next Generation Impactor instrument (NGI). The soft mist inhaler was held close to the NGI inlet until no aerosol was visible. The flow rate of the NGI was set to 30 L/minute and was operated under ambient temperature and a relative humidity (RH) of 90±2%.

Sample VI was discharged into the NGI. Fractions of the dose were deposited at different stages of the NGI, in accordance with the particle size of the fraction. Each fraction was washed from the stage and analyzed using HPLC.

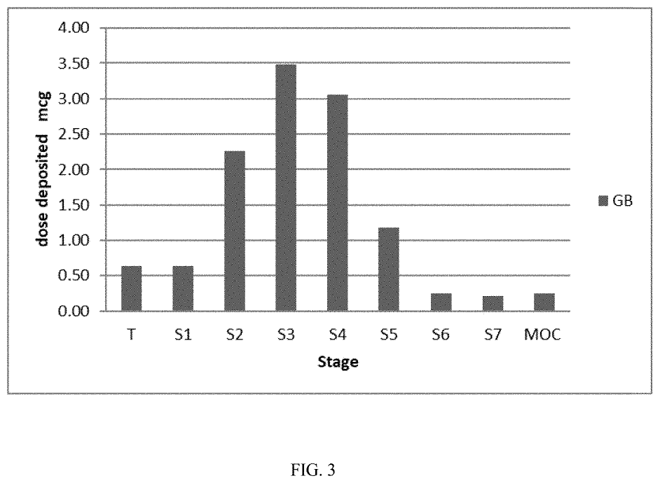

The particle size distribution was expressed in terms of mass median aerodynamic diameter (MMAD) and geometric standard deviation (GSD). The results showed that the MMAD of glycopyrronium bromide was less than about 6 μm, and the GSD of glycopyrronium bromide was less than about 2. The results are provided in Table 9 below. The aerodynamic particle size distribution of sample VI was investigated by NGI, and the results are shown in FIG. 3.

TABLE 9
Aerodynamic Particle Size Distribution

| Particle size parameter | Glycopyrrolate |
|---|---|
| Fine particle dose (FPD) | 8.44 μg |
| Fine Particle Fraction (FPF) | 70.45% |
| MMAD (μm) | 4.05 |
| GSD | 1.77 |

### Example 8

Stability Experiment:

Samples VII, VIII, and IX were prepared as follows:

50% benzalkonium chloride aqueous solution and edetate disodium dihydrate according to the amounts provided in Table 10 were dissolved in 190 ml purified water, and the resulting solutions were adjusted to the target pH values with citric acid (CA). Glycopyrronium bromide (GB) according to Table 10 was added to each solution, and the resulting mixtures were stirred until completely dissolved. Finally, purified water was added to each sample to a final volume of 200 ml, and each solution was mixed well.

TABLE 10
Ingredient Contents of Samples VII-IX of
200 ml Inhalation Solution Formulation

| | | |
|---|---|---|
| Sample VII | Sample VIII | Sample IX |

| | | | | | | |
|---|---|---|---|---|---|---|
| GB | 226.22 | mg | 226.20 | mg | 226.21 | mg |
| 50% BAC | 40.34 | mg | 40.04 | mg | 40.34 | mg |
| EDTA | 20.03 | mg | 20.04 | mg | 20.03 | mg |

| | | | |
|---|---|---|---|
| CA•H2O | Adjust to | Adjust to | Adjust to |
| | pH 2.5 | pH 3.0 | pH 3.4 |
| Purified | Added to | Added to | Added to |
| Water | 200.00 ml | 200.00 ml | 200.00 ml |

The obtained solutions were filled into LDPE containers and sealed with aluminum foil, and stored at 40° C.±2° C./75%±5% RH.

The impurity analysis method is as follows:

### Column: Inersil-ODS-3, 150×460 mm, S-5.0 m, 12 nm

Column temperature: 40° C.; Flow: 1.0 mL/min; Volume: 10 μL; Detection wavelength: 210 nm;
Run time: 60 minutes.
Mobile phase A: 1.00 g sodium hexane sulfonate, 1.00 g anhydrous sodium sulfate dissolved in 650 mL water, add 0.5 mol/L sulfuric acid 2 ml, and the buffer salt solution obtained after mixing.
Mobile phase B: Acetonitrile.

| Time (min) | Mobile phase A % | Mobile phase A % |
|---|---|---|
| 0 | 70 | 30 |
| 10 | 65 | 35 |
| 15 | 60 | 40 |
| 25 | 55 | 45 |
| 30 | 50 | 50 |
| 50 | 30 | 70 |
| 51 | 70 | 30 |
| 60 | 70 | 30 |

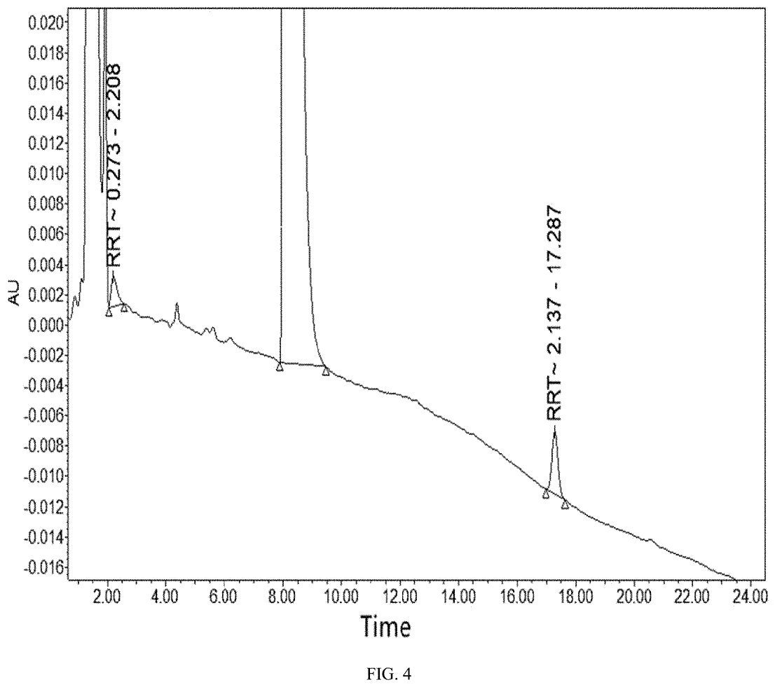

Impurities are analyzed according to the above analysis method. The stability data is shown in Tables 11-13 below. The relative retention time of the unknown maximum impurity is 0.273. The relative retention time of impurity GB-J is 2.137. FIG. 4 shows an HPLC trace demonstrating the relative retention times of the unknown maximum impurity and impurity GB-J.

TABLE 11
The Stability Results of Sample VII (conditions: 40°
C. ± 2° C./75% ± 5% RH)

| | | Sample VII | | | |
|---|---|---|---|---|---|
| | | Target pH | | | |
| | | 2.5 | | | |
| | | Time | | | |
| | | 0 | 1 | 3 | 6 |
| | | days | month | months | months |
| Measured pH | | 2.58 | 2.57 | 2.59 | 2.62 |
| GB concentration | | 114.83 | 112.8 | 112.7 | 111.5 |
| (mg/100 ml) | | | | | |
| GB content (%) | | 101.52 | 99.72 | 99.63 | 98.61 |
| 50% BAC | | 20.42 | 19.9 | 20.57 | 20.25 |
| concentration | | | | | |
| (mg/100 ml) | | | | | |
| 50% BAC content (%) | | 101.25 | 98.67 | 102 | 100.4 |
| EDTA concentration | | 10.08 | 9.78 | 9.98 | 9.94 |
| (mg/100 ml) | | | | | |
| EDTA content (%) | | 100.7 | 97.66 | 99.78 | 99.45 |
| impurity | GB-J | ND | 0.09 | 0.04 | 0.51 |
| (%) | RRT = 0.273 | ND | ND | ND | 0.15 |
| | Total | ND | 0.09 | 0.04 | 0.66 |
| | impurities | | | | |

ND: not detected

TABLE 12
The Stability Results of Sample VIII (conditions: 40°
C. ± 2° C./75% ± 5% RH)

| | | Sample VIII | | | |
|---|---|---|---|---|---|
| | | Target pH | | | |
| | | 3 | | | |
| | | Time | | | |
| | | 0 | 1 | 3 | 6 |
| | | days | month | months | months |
| Measured pH | | 3.02 | 3 | 3.04 | 3.06 |
| GB concentration | | 114.3 | 112.6 | 112.7 | 114.4 |
| (mg/100 ml) | | | | | |
| GB content (%) | | 101.1 | 99.57 | 99.65 | 101.2 |
| 50% BAC | | 20.13 | 20.22 | 20.6 | 20.61 |
| concentration | | | | | |
| (mg/100 ml) | | | | | |
| 50% BAC content (%) | | 100.5 | 101 | 102.9 | 103 |
| EDTA concentration | | 10.13 | 9.77 | 10.13 | 10.11 |
| (mg/100 ml) | | | | | |
| EDTA content (%) | | 101.1 | 97.47 | 101.28 | 101.1 |
| Impurity | GB-J | ND | 0.05 | 0.02 | 0.32 |
| (%) | RRT = 0.273 | ND | ND | ND | 0.25 |
| | Total | ND | 0.05 | 0.02 | 0.57 |
| | impurities | | | | |

ND: not detected

TABLE 13
The Stability Results of Sample IX (conditions: 40°
C. ± 2° C./75% ± 5% RH)

| | | Sample IX | | | |
|---|---|---|---|---|---|
| | | Target pH | | | |
| | | 3.5 | | | |
| | | Time | | | |
| | | 0 | 1 | 3 | 6 |
| | | days | month | months | months |
| Measured pH | | 3.42 | 3.39 | 3.43 | 3.43 |
| GB concentration | | 117.43 | 112.01 | 113.25 | 112.65 |
| (mg/100 ml) | | | | | |
| GB content (%) | | 104 | 99.03 | 100.13 | 99.6 |
| 50% BAC | | 20.12 | 20.03 | 20.29 | 20.37 |
| concentration | | | | | |
| (mg/100 ml) | | | | | |
| 50% BAC content (%) | | 99.77 | 99.31 | 100.57 | 100.98 |
| EDTA concentration | | 10.15 | 9.81 | 10.03 | 10.03 |
| (mg/100 ml) | | | | | |
| EDTA content (%) | | 101.38 | 97.92 | 100.26 | 100.32 |
| Impurity | GB-J | ND | 0.06 | 0.03 | 0.35 |
| (%) | RRT = 0.273 | ND | ND | ND | 0.18 |
| | Total | ND | 0.06 | 0.03 | 0.53 |
| | impurities | | | | |

ND: not detected

As shown in Tables 11, 12, and 13, at pH 2.5-3.5 glycopyrronium bromide solutions show increased stability. Glycopyrronium bromide solutions ranging from a pH of about 2.5 to about 3.5 are stable for about 6 months at 40° C.±2° C./75%±5% RH.

### Example 9

Comparison Test of Atomization Effect of Different Devices:

The atomization effects of three devices have been compared: 1. The soft mist inhaler disclosed in U.S. 2019/0030268; 2. An LC-PLUS air compression atomization device; 3. A GB powder aerosol device.

1. The inhaler disclosed in U.S. 2019/0030268 is made by ourselves.

2. The LC-PLUS air compression atomization device is model Pari TurboBoY, purchased from Pari.

3. The GB powder aerosol device is model breezhaler, purchased from NOVARTIS.

Administration Using a Soft Mist Inhalation Device

TABLE 14
Ingredient Contents of Sample X of 100 ml Inhalation Solution
Formulation For Administration by Soft Mist Inhalation

| | Sample X | |
|---|---|---|
| Ingredients | Theoretical dose | |
| GB | 160.00 | mg |
| 50% BAC | 20.00 | mg |
| EDTA | 10.00 | mg |
| CA | 20.00 | mg |

| | |
|---|---|
| water | Add to 100.00 ml |
| Target pH | 3.20 |

Sample X inhalation solution for administration by soft-mist inhalation was prepared as follows:

50% benzalkonium chloride aqueous solution and edetate disodium dihydrate according to Table 14 were dissolved in 95 ml purified water, and the solution was adjusted to the target pH with anhydrous citric acid (CA). Glycopyrronium bromide according to Table 14 was added to the solution, and then the mixture was sonicated until completely dissolved. Finally, purified water was added to a final volume of 100 ml.

The aerodynamic particle size distribution was determined using a Next Generation Impactor instrument (NGI). The soft mist inhaler is disclosed in U.S. 2019/0030268. The soft mist inhaler was held close to the NGI inlet until no aerosol was visible. The flow rate of the NGI was set to 30 L/minute and was operated under ambient temperature and a relative humidity (RH) of 90±2%.

Sample X was discharged into the NGI. Fractions of the dose were deposited at different stages of the NGI, in accordance with the particle size of the fraction. Each fraction was washed from the stage and analyzed using HPLC. The results are provided in Table 15 below.

TABLE 15
Single Dose Level Distribution and Aerodynamic Particle
Size Distribution of GB Inhalation Formulation Sample
X (1.60 mg/ml) Administered by Soft Mist Inhalation

| | Dosage | Percentage content | Cut-off diameter |
|---|---|---|---|
| GB Deposited | (μg) | at all levels | (μm) |
| Throat | 7.64 | 21.67% | |
| Stage 1 | 1.23 | 3.48% | 11.72 |
| Stage 2 | 5.29 | 14.99% | 6.4 |
| Stage 3 | 8.99 | 25.51% | 3.99 |
| Stage 4 | 7.28 | 20.64% | 2.3 |
| Stage 5 | 2.86 | 8.11% | 1.36 |
| Stage 6 | 1.19 | 3.38% | 0.83 |
| Stage 7 | 0.33 | 0.94% | 0.54 |
| Micro-Orifice | 0.45 | 1.29% | |
| Collector (MOC) | | | |
| Theoretical dose(μg) | 35.36 | | |
| Actual test dose(μg) | 35.26 | | |
| Recovery rate % | 99.72% | | |
| Fine Particle Fraction | 59.86% | | |
| (FPF) | | | |

Fine Particle Fraction (FPF) is the proportion of fine particle dose in the released dose.

$$F P F = \frac{Mass < 5 μm}{Mass Total dose} .$$

The larger the FPF value, the higher the atomization efficiency.

Administration Using an LC-PLUS Air Compression Atomization Device

TABLE 16
Ingredient Contents of Sample XI of 100 ml Inhalation
Solution Formulation for Administration by the
LC-PLUS Air Compression Atomization Device

| | Sample XI | |
|---|---|---|
| Ingredients | Theoretical dose | |
| GB | 2.50 | mg |
| CA | 384.00 | mg |
| NaCl | 780.00 | mg |

| | |
|---|---|
| NaOH | Adjust pH to 3.70 |
| water | Add to 100.00 ml |

Sample XI inhalation solution for administration by the LC-PLUS air compression atomization device was prepared as follows:

NaCl and CA according to Table 16 were dissolved in 95 ml purified water, and the solution was adjusted to the target pH with NaOH. Glycopyrronium bromide according to Table 16 was added to the solution, and then the mixture was sonicated until completely dissolved. Finally, purified water was added to a final volume of 100 ml.

The aerodynamic particle size distribution was determined using a Next Generation Impactor instrument (NGI). The atomization device is the LC-PLUS air compression atomization device. The flow rate of the NGI was set to 15 L/minute and was operated under ambient temperature and a relative humidity (RH) of 90±2%.

Sample XI was discharged into the NGI. Fractions of the dose were deposited at different stages of the NGI, in accordance with the particle size of the fraction. Each fraction was washed from the stage and analyzed using HPLC. The results are provided in Table 17 below.

TABLE 17
Single Dose Level Distribution And Aerodynamic
Particle Size Distribution of GB Inhalation Formulation
Sample XI (0.025 mg/ml) Administered by An LC-PLUS
Air Compression Atomization Device

| | Dosage | Percentage content | Cut-off Diameters |
|---|---|---|---|
| GB Deposited | (μg) | at all levels | (μm) |
| LC Plus device | 16.5 | 59.37% | |
| Throat | 9.59 | 34.53% | |
| Stage 1 | 0.00 | 0.00% | 14.1 |
| Stage 2 | 0.07 | 0.24% | 8.61 |
| Stage 3 | 0.12 | 0.44% | 5.39 |
| Stage 4 | 0.36 | 1.31% | 3.3 |
| Stage 5 | 0.51 | 1.83% | 2.08 |
| Stage 6 | 0.34 | 1.22% | 1.36 |
| Stage 7 | 0.22 | 0.80% | 0.98 |
| MOC | 0.07 | 0.27% | 0 |
| Theoretical dose | 25.00 | | |
| (μg) | | | |
| Actual test dose | 27.79 | | |
| (μg) | | | |
| Recovery rate % | 111.15% | | |
| FPF | 5.43% | | |

Table 17 shows that the fine particle fraction (FPF) is only 5.43%, which is far lower than the FPF value using the soft mist inhaler of the present invention. When the LC-PLUS air compression atomization device is used to atomize the GB solution, a large amount of drug residue remains inside the device and the simulated throat. The medicine remaining inside the device and throat cannot reach the lungs to produce therapeutic effects. The experimental results show that the formulation of the present invention is more effectively atomized by the soft mist device of the present invention than by the LC Plus device.

Administration Using a GB Powder Aerosol Device

The GB powder aerosol device and capsule are purchased from NOVARTIS. The model is called breezhaler.

The aerodynamic particle size distribution was determined using a Next Generation Impactor instrument (NGI). The atomization device is the GB powder aerosol device, and the formulation is the glycopyrronium bromide capsule purchased from NOVARTIS. The flow rate of the NGI was set to 90 L/minute and was operated under ambient temperature and a relative humidity (RH) of 90±2%.

Each capsule contains 63 mcg GB. The capsule was discharged into the NGI. Fractions of the dose were deposited at different stages of the NGI, in accordance with the particle size of the fraction. Each fraction was washed from the stage and analyzed using HPLC. The results are provided in Table 18 below.

TABLE 18
Layer Distribution And Aerodynamic Particle Size Distribution
of GB Powder Aerosol Administered by A Powder Aerosol Device

| | Dosage | Percentage content | Cut-off Diameter |
|---|---|---|---|
| GB | (μg) | at all levels | (μm) |
| capsule | 17.13 | 28.71% | |
| device | 1.78 | 2.98% | |
| Pre-separator | 7.02 | 11.76% | |
| Throat | 6.73 | 11.28% | |
| Stage 1 | 3.02 | 5.06% | 6.48 |
| Stage 2 | 8.58 | 14.38% | 3.61 |
| Stage 3 | 8.68 | 14.55% | 2.3 |
| Stage 4 | 5.12 | 8.58% | 1.37 |
| Stage 5 | 1.61 | 2.70% | 0.76 |
| Stage 6 | 0 | 0.00% | 0.43 |
| Stage 7 | 0 | 0.00% | 0.26 |
| MOC | 0 | 0.00% | 0 |
| Theoretical dose | 63 | | |
| Actual test dose (μg) | 59.67 | | |
| Recovery rate % | 94.71% | | |
| FPF | 40.20% | | |

Table 18 shows that the fine particle fraction (FPF) is only 40.20%, which is far lower than the FPF value using the soft mist inhaler of the present invention. When a powder aerosol device is used to atomize capsules a large amount of drug residue remains in the capsule, device, pre-separator, and throat, resulting in a large amount of drugs that are not effectively atomized, and a large amount of drugs do not reach the lungs. The atomization efficiency of this dosage form is not high. The required dosage will be much higher than that of the GB solution of the present invention using a soft mist inhalation device. The experimental results show that the formulation of the present invention is more effectively atomized by the soft mist device of the present invention than by the powder aerosol device.

The GB solution formulation of the present invention uses a soft mist device, which has the characteristics of efficient atomization. At the same effective concentration, the GB solution formulation of the present invention may be administered at a lower dose than the capsule powder aerosol produced by Novartis or than a formulation administered by an LC-PLUS air compression atomization device.

While various embodiments of the present invention have been described above, it should be understood that they have been presented by way of example only and not limitation. For example, the present invention is not limited to the physical arrangements or dimensions. Nor is the present invention limited to any particular design or materials of construction. As such, the breadth and scope of the present invention should not be limited to any of the above-described exemplary embodiments, but should be defined only in accordance with the following claims and their equivalents.

What is claimed is:

1. A liquid, propellant-free pharmaceutical formulation comprising: (a) glycopyrronium or a salt thereof present in an amount between about 10 mg/100 ml and about 500 mg/100 ml; (b) water; (c) a pharmacologically acceptable stabilizer; and (d) a pharmacologically acceptable preservative, wherein the pharmaceutical formulation has a pH in a range from about 2.0 to about 6.0, and wherein the formulation is administered using a soft mist inhaler.

2. The pharmaceutical formulation of claim 1, wherein the pharmaceutical formulation has a pH in a range from about 2.0 to 5.0.

3. The pharmaceutical formulation of claim 2, wherein the pharmaceutical formulation has a pH in a range from about 2.5 to 4.0.

4. The pharmaceutical formulation of claim 1, wherein the glycopyrronium or a salt thereof is glycopyrromium bromide.

5. The pharmaceutical formulation of claim 1, wherein the pharmacologically acceptable preservative is selected from the group consisting of benzalkonium chloride, benzoic acid, sodium benzoate, and a combination thereof.

6. The pharmaceutical formulation of claim 1, wherein the preservative is present in an amount ranging from about 2 mg/100 ml to about 1000 mg/100 ml.

7. The pharmaceutical formulation of claim 1, wherein the stabilizer is selected from the group consisting of edetic acid, edetate disodium dehydrate, edetate disodium, citric acid, and any combination thereof.

8. The pharmaceutical formulation of claim 1, wherein the stabilizer is present in an amount ranging from about 2 mg/100 ml to about 500 mg/100 ml.

9. The pharmaceutical formulation of claim 1, wherein the pharmaceutical formulation comprises a pharmacologically acceptable additive.

10. A method for administering a pharmaceutical formulation of claim 1 to a patient, comprising forming an inhalable aerosol by using pressure to force a defined amount of the pharmaceutical formulation through a nozzle to nebulize the pharmaceutical formulation.

11. The method of claim 10, wherein the defined amount of the pharmaceutical formulation ranges from about 5 to about 30 microliters.

12. The method of claim 11, wherein the inhalable aerosol has an aerosol D50 of less than about 15 μm.

13. The method of claim 11, wherein the pharmaceutical formulation is administered using an inhaler comprising a blocking function and a counter.

14. A method of treating asthma or COPD in a patient, comprising administering to the patient the pharmaceutical formulation of claim 1.

15. A method of treating asthma or COPD in a patient, comprising administering to the patient the pharmaceutical formulation using the method of claim 11.

16. A method for administering a pharmaceutical formulation of claim 1, comprising nebulizing the pharmaceutical formulation in an inhaler, wherein the inhaler includes a blocking function and a counter.

17. The method of claim 12, wherein the patient has asthma or COPD.

18. A device for administering glycopyrronium or a salt thereof comprising:
(a) a soft mist inhaler and
(b) a liquid, propellant-free pharmaceutical formulation comprising:
(i) glycopyrronium or a salt thereof present in an amount between about 20 mg/100 ml and about 500 mg/100 ml;
(ii) water;
(iii) a pharmacologically acceptable stabilizer; and
(iv) a pharmacologically acceptable preservative,
wherein the pharmaceutical formulation has a pH in a range from about 2.0 to about 6.0, and
wherein the formulation is contained in the soft mist inhaler.
